(19) United States
(12) Patent Application Publication
(10) Pub. No.: US 2008/0175799 A1
(43) Pub. Date: Jul. 24, 2008

(54) Dentifrice Compositions Comprising as a Soluble Calcium Sequestering Agent a Derivative Comprising a Phosphonate Group

(75) Inventor: Jonathan Edward Creeth, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXOSMITHKLINE RESEARCH AND DEVELOPEMENT LIMITED, Brentford, Middlesex (GB)

(21) Appl. No.: 11/909,207

(22) PCT Filed: Mar. 17, 2006
(86) PCT No.: PCT/EP06/02667

§ 371 Date: Sep. 20, 2007

(30) Foreign Application Priority Data
Mar. 21, 2005 (GB) 0505758.3

(51) Int. Cl. A61K 8/18; A61Q 11/00

(52) U.S. Cl. 424/49

(57) ABSTRACT

A dentifrice composition is disclosed comprising a soluble calcium sequestering agent that is not an oxidising agent, wherein the soluble calcium sequestering agent is a polymeric material comprising a phosphonate group. The composition has an RDA value of below 30 and an IVSR value greater than 50 (when compared to a Control) and an orally acceptable vehicle, wherein the calcium sequestering agent is present in a proportion of 1-20 wt % and an abrasive is present in a proportion of 0-5 wt % of the composition. The composition is highly effective in removing stains from natural teeth and prosthetics and will not damage the tooth or prosthetic due to excessive abrasion. The compositions are also useful for combating the problems associated with acid erosion and/or tooth wear.

The present invention relates to dentifrice compositions, in particular compositions comprising a fluoride source and a soluble calcium sequestering agent that is not an oxidising agent, for cleaning natural teeth and dentures. Such compositions show excellent cleaning properties whilst at the same time low abrasion characteristics. The compositions are also useful for combating the problems associated with acid erosion and/or tooth wear

Dentifrices have been used for more than 2000 years and the principal purpose of a dentifrice has always been the removal of surface deposits from the teeth. It is known that removal of surface deposits from the teeth can be achieved through the use of either a chemical or mechanical cleaning agent. An example of a chemical cleaning agent is a calcium-sequestering agent, for example a polyphosphate salt, such as tripolyphosphate, and the mechanical agents are the abrasive materials, e.g. precipitated silica or calcium carbonate. An example of an abrasive cleaning agent is amorphous hydrated silica. Bleaching agents are sometimes included in dentifrice compositions to decolourise tooth stains. This will not however remove the stain effectively and, furthermore, effective bleaching agents can damage the oral tissues and are difficult to formulate successfully. Hydrogen peroxide is a well-known bleaching agent but with peroxide-containing dentifrices the lifetime of peroxide in the oral cavity is very short. Human plaque contains a significant level of the peroxide-degrading enzyme catalase, which means that it is very difficult to maintain an effective level of peroxide on the tooth surface for sufficient time for stain to be bleached. Use of more aggressive bleaching agents than peroxides however bring concerns over tissue damage.

Traditional dentifrice formulations contain a number of specific components for example abrasive agents, humectants, fluoride sources, binders, anti plaque agents, dyes, flavours, preservatives, water and other optional ingredients. Fluoride is an important aid to oral health, particularly for the health of the tooth's enamel. The effective removal of surface deposited stains from the teeth or a dental prosthesis is always associated with substantially abrasive formulations. It is however recognised that dentifrice compositions containing abrasive materials can damage the tooth surface as well as the surface of dental prosthetics so it is very important that a dentifrice formulation should provide effective cleaning without subjecting the tooth or prosthetic to excessive abrasion.

WO-A-95/17158 describes and claims a composition for reducing or removing surface deposited stains from natural teeth or dental prostheses comprising a dentally acceptable preparation comprising 5 to 15% by weight of a water soluble alkali metal tripolyphosphate. All dentifrice formulation examples shown in WO-A-95/17158 include conventional dental abrasives, for example abrasive silica, is disclosed in the range 5-80 wt. %.

The established method for determining the abrasivity of a dentifrice formulation is by measuring the Relative Dentine Abrasivity (RDA) (Hefferen, J J. A laboratory method for measuring dentifrice abrasivity. J. Dent. Res. 55 563-573, 1976.). This assay measures loss of dentine due to extended brushing with a 25:40 w/w slurry of test toothpaste from prepared samples of human dentine. The dentine samples are irradiated to generate ³²P in the mineral. The assay measures radioactivity in the supernatant after brushing, relative to radioactivity liberated by brushing with a standard slurry of calcium pyrophosphate.

Cleaning performance is closely linked to stain removal and can be measured in various ways. There is a very useful published in-vitro stain removal (IVSR) assay (Layer T M, McConville P S and Wicks M A. Stain removal efficacy of whitening toothpastes—in vitro studies. J. Dent. Res. 79: 216 abstract 581, 2000) that is used in the present invention. This assay aims to maximise the relevance of the assay to the in vivo situation by using untreated naturally stained bovine teeth as substrate. The teeth are brushed for an extended period with a 1:3 slurry of test toothpaste in water. Stain removal is quantified using a chromameter. Performance may be measured relative to a Control dentifrice formulation containing 14% Zeodent 113™ abrasive silica (available from J.M. Huber Corporation) in a conventional base containing water, sorbitol, glycerin, PEG, flavour, SLS, sodium saccharin, Xanthum gum and sodium fluoride. IVSR is also known as the natural extrinsic stain removal assay or NESR and is referred to as such in some publications.

Cleaning efficiency may therefore be described as the ratio of the IVSR to the RDA.

IVSR values that are quoted herewith are relative to a control formulation that has been assigned an arbitrary value of 100.

The efficacy of dentifrices of the present invention in combating dental erosion can be assessed by measuring their ability to reduce the solubility of enamel when treated with lactic acid using a standard FDA method 33 for sodium fluoride silica dentifrices. This is know as the Enamel Solubility Reduction Assay.

EP 0 835 223B discloses an amorphous silica that has a low abrasivity value which, when incorporated into a dentifrice composition, maintains good cleaning characteristics. This publication however considers the abrasive silica as the only cleaning material of the composition and describes the amorphous silica in terms of its physical properties. Accordingly EP 0 835 223B claims an amorphous silica characterized by: an RDA value of between 30 and 70, an oil absorption capacity of between 100 and 155 cm³/100 g, and a BET surface area of up to 200 m²/g.

From the literature it appears that many formulations that are dependent for their cleaning properties upon their abrasive content are described and claimed in terms of the physical properties of the abrasive material. For example EP-A-0 396 460 describes in Example 1 an amorphous silica in terms of BET surface area, oil uptake, pore volume, pH, refractive index and translucence.

EP-A-0 002 184 discloses the use of a sodium polyphosphate in fine granulate form for tooth cleaning, either by itself or in combination with a commercial toothpaste formulation, referring to the abrasive effect of the sodium polyphosphate and intensification of tooth cleaning by this material without damaging the substance of the teeth.

A highly effective dentifrice formulation without the traditional amounts of abrasive material has been described in PCT/EP2004/010629. In this application calcium sequestering agents, such as sodium tripolyphosphate are described in formulations that have no or very low levels of abrasive material. Such compositions have good cleaning characteristics with an abrasivity (measured as RDA) of below 30.

The present invention concerns the use of other calcium sequestering agents, such as polymeric materials that comprise a phosphonate group.

It is an object of the present invention to provide a dentifrice composition comprising as a soluble calcium sequestering agent that is not an oxidising agent, a polymeric material comprising a phosphonate group wherein the composition has an RDA value of below 30 and an IVSR value greater than 50 (when compared to a Control) and an orally acceptable vehicle, wherein the calcium sequestering agent is present in a proportion of 1-20 wt % and an abrasive is present in a proportion of 0-5 wt % of the composition.

Preferably the composition is a toothpaste being a viscous extrudable fluid which can be provided in a collapsible container e.g. a tube or a pump and extruded therefrom onto a toothbrush for use.

Preferably the RDA value is below 25, preferably below 20, and most preferably as low as possible, e.g. so that the composition is substantially non-abrasive. Preferably the IVSR value is up to 250, preferably 200 and more preferably greater than 100, for example being in the range 75-150, e.g. 75-120.

Examples of the polymeric material comprising a phosphonate group include those described and referred to in published patent application WO 2004/054529 (The Procter+Gamble Company). The polymeric material may also be mixed with other calcium sequestering agents such as polyphosphates as described and referred to in PCT/EP2004/010629.

Preferred polymeric materials comprising a phosphonate group are azacycloheptane diphosphonate and ethanehydroxy bisphosphonate.

When high levels of soluble calcium-sequestering agents are used in a formulation, there is often a certain amount of undissolved solids in the formulation. Any undissolved calcium-sequestering agent in compositions of the present invention will thus during use dissolve rapidly and provide a cleaning effect in its dissolved state.

The soluble calcium-sequestering agent may be present in a proportion 1-20 wt %, preferably 2-15 wt%, more preferably 5-15 wt% of the dentifrice composition. By using a proportion of the calcium-sequestering agent in the composition below the solubility limit thereof a gel or liquid compositions may be provided in which the calcium sequestering agent is in solution, so that the gel or liquid may include no undissolved solid particles, and may be a clear gel or liquid.

The composition may include a fluoride ion source. Fluoride ion may stabilise polyphosphates in the mouth. For a composition for use in cleaning natural teeth the inclusion of a fluoride ion source is also desirable because of its caries protection activity, but for a composition for use in cleaning artificial dentures such activity is not necessary. The fluoride ion source may be provided by an alkali metal fluoride, preferably sodium fluoride, an alkali metal monofluorophosphate, stannous fluoride and the like. Preferably, however, the fluoride ion source is an alkali metal fluoride, most preferably sodium fluoride. The fluoride ion source serves in a known manner for caries protection. Preferably, the fluoride ion source will be used in an amount to provide an anti-caries effective amount and a phosphatase enzyme inhibiting amount, such as an amount sufficient to provide from about 25 ppm to about 3500 ppm, preferably about 1100 ppm, as fluoride ion. For example the formulation may contain 0.1-0.5 wt % of an alkali metal fluoride such as sodium fluoride.

Preferably the pH of the composition is from 6 to 10.5, more preferably from 7 to 9.5. Typically the composition may contain up to 0.5 wt. % of sodium hydroxide to provide a suitable pH.

In compositions of the present invention i.e. those which can be extruded onto a toothbrush, the orally acceptable vehicle may be of a generally conventional composition e.g. comprising a thickening agent, a binding agent and a humectant. Preferred binding agents include for example natural and synthetic gums such as xanthan gums, carageenans, alginates, cellulose ethers and esters. Preferred humectants include glycerin, sorbitol, propylene glycol and polyethylene glycol. A preferred humectant system consists of glycerin, sorbitol and polyethylene glycol.

In addition, the orally acceptable vehicle may optionally comprise one or more surfactant, sweetening agent, flavouring agent, anticaries agent (in addition to the fluoride ion source), anti-plaque agent, anti-bacterial agent such as triclosan or cetyl pyridinium chloride, tooth desensitizing agent, colouring agents and pigment. Useful surfactants include the water-soluble salts of alkyl sulphates having from 10 to 18 carbon atoms in the alkyl moiety, such as sodium lauryl sulphate, but other anionic surfactants as well as non-ionic, zwitterionic, cationic and amphoteric surfactants may also be used.

If an aqueous orally acceptable vehicle is employed, a toothpaste composition of the present invention suitably contains from about 10 to about 80 wt % humectant such as sorbitol, glycerin, polyethylene glycol or xylitol; from about 0.25 to about 5 wt % detergent; from 0 to about 2 wt % sweetener and flavouring agents; together with water and an effective amount of binding and thickening agents, such as from about 0.1 to about 15 wt %, to provide the toothpaste of the invention with the desired stability and flow characteristics.

It is preferred to use a thickening silica as the thickening agent. So called “thickening silicas” are known silicas which have relatively little abrasive effect compared with known abrasive silicas such as Zeodent 113™, but provide a thickening effect on the composition. Suitable thickening silicas are known and include those marketed by Huber under the tradename Zeodent, e.g. Zeodent 167, by Degussa AG under the trade name SIDENT®, e.g. SIDENT 22S®, and by Grace-Davison Chemical Division under the trade name SYLOBLANC®, e.g. SYLOBLANC 15®, respectively. For example the composition may contain up to ca. 20 wt % of a thickening silica, typically 5-15 wt %.

The composition of the invention may contain an abrasive material, for example known types of “abrasive silica” commonly used in toothpaste compositions e.g. Zeodent 113™ as mentioned above. However to achieve the RDA below 30 it is preferred to include as little of this type of abrasive material as possible, preferably less than 5 wt. %, more preferably less than 2 wt. % abrasive, most preferably 0 wt. % abrasive material, in addition to any mild abrasive effect produced by other solid particles in the composition, e.g. undissolved calcium sequestering agent and e.g. any thickening silica present. For the purposes of this invention an abrasive material may be defined as a material having an RDA of 30 or above. Larger amounts of silica may be incorporated, more typical of abrasive silica incorporation levels in toothpastes (up to e.g. 25%), if that silica has an RDA below 30.

Regarding the RDA values given in the preceding paragraph, it should be noted that the slurry conditions used to determine the RDA of an abrasive raw material differ from those used to determine the RDA of a formulated toothpaste. The slurry conditions for a raw material are 10 g abrasive plus 50 ml of a 0.5% carboxymethyl cellulose slurry in 10% glycerin, whereas the slurry conditions for a formulated toothpaste are 25 g toothpaste plus 40 ml water. Care should therefore be taken when comparing RDA figures for abrasives as raw materials with RDA figures for fully formulated toothpastes.

Therefore a preferred dentifrice composition of this invention comprises as a soluble calcium sequestering agent that is not an oxidising agent, a polymeric material comprising a phosphonate group wherein the composition has an RDA value of below 30 and an IVSR value greater than 50 (when compared to a Control) and an orally acceptable vehicle, wherein the calcium sequestering agent is present in a proportion 1-20 wt %, preferably 5-10 wt % of the composition, with 0-5 wt %, preferably 0 wt % of an abrasive, typically an abrasive silica.

The dentifrice composition may be presented as either a single or dual phase composition.

Suitably the composition is in the form of a conventional toothpaste-type composition that can be squeezed from a collapsible tube. It is also suitable for dispensing from a pressurised aerosol container.

Toothpaste-type compositions according to the present invention may be prepared by admixing according to conventional practice the calcium sequestering agent, and the fluoride ion source if present, with the orally acceptable dental vehicle, which may be anhydrous but is preferably an aqueous orally acceptable dental vehicle, to form a storage-stable semi-solid extrudable material useful as a dentifrice. The composition of the invention will now be described by way of non-limiting examples only.

TABLE 1
Examples of the present invention

| Ingredient | Control | 1 |
|---|---|---|
| Glycerin, 98% min | 10.00 | 11.20 |
| Sorbitol, 70% soln. | 26.00 | 29.11 |
| Peg 6 | 3.00 | 3.00 |
| Xanthan gum | 1.20 | 0.90 |
| Flavour | 1.00 | 1.00 |
| Soluble saccharin | 0.21 | 0.21 |
| Sodium lauryl sulphate | 1.15 | 1.15 |
| azacycloheptane | 0.00 | 5.00 |
| diphosphonate | | |
| Sodium hydroxide | 0.00 | 0.00 |
| Sodium fluoride | 0.24 | 0.24 |
| Titanium dioxide | 1.00 | 1.45 |
| Abrasive silica Zeodent | 14.00 | 0.00 |
| 113 | | |
| Thickening silica | 9.00 | 13.50 |
| De-ionised water | 33.20 | 32.24 |
| Total | 100.00 | 100 |

Example 1 is a formulation in which the calcium-sequestering chemical cleaning system is azacycloheptane diphosphonate. Thickening silica is increased to account for the lower level of solids in the formulation. Other calcium sequestering agents may be used as chemical cleaning agent and/or anticaries agent.

1. A dentifrice composition comprising as a soluble calcium sequestering agent that is not an oxidising agent, a polymeric material comprising a phosphonate group wherein the composition has an RDA value of below 30 and an IVSR value greater than 50 (when compared to a Control), and an orally acceptable vehicle, wherein the calcium sequestering agent is present in a proportion of 1-20 wt % and an abrasive is present in a proportion of 0-5 wt % of the composition.

2. A dentifrice composition according to claim 1, further comprising an abrasive silica that has an RDA below 30.

3. A dentifrice composition according to claim 2, wherein the RDA value is below 20 and the IVSR value is up to 250.

4. A dentifrice composition according to claim 3, wherein the calcium sequestering agent is present in an amount of from 2-15 wt % of the composition.

5. A dentifrice composition according to claim 4, wherein 0 wt % of the abrasive is present in the composition.

6. A dentifrice composition according to claim 5, wherein the IVSR value is measured relative to a Control dentifrice containing 14% Zeodent 113 abrasive silica in a conventional base containing water, sorbitol, glycerin, PEG, flavour, SLS, sodium saccharin, Xanthum gum and sodium fluoride.

7. (canceled)

8. A dentifrice composition according to claim 1 in the form of a viscous extrudable fluid which can be provided in a collapsible container.
